(19) United States
(12) Patent Application Publication
(10) Pub. No.: US 2009/0143493 A1
(43) Pub. Date: Jun. 4, 2009

(54) CATALYST AND METHOD

(76) Inventors: Jacobus Johannes Cornelis Geerlings, Amsterdam (NL); Marinus Johannes Reynhout, Amsterdam (NL); Wilhelmus Johannes Scholten, Amsterdam (NL); Guy Lode Verbist, Amsterdam (NL)

Correspondence Address: SHELL OIL COMPANY, P O BOX 2463, HOUSTON, TX, 772522463, US

(21) Appl. No.: 12/244,415

(22) Filed: Oct. 2, 2008

(30) Foreign Application Priority Data
Oct. 4, 2007 (EP) PCT/EP2007/060524

(51) Int. Cl. C07C 1/02; B32B 5/16

(52) U.S. Cl. 518/715; 428/402; 977/775

(57) ABSTRACT

A titania catalyst support having a particle size distribution with a first peak at a first particle size and a second peak at a second particle size, wherein the second particle size is at least 50% larger than the first particle size. A method of manufacture is also disclosed. The support and resulting catalyst can be used for catalysing a Fischer-Tropsch reaction.

This application claims the benefit of application PCT/EP2007/060524 filed Oct. 4, 2007.

### BACKGROUND OF THE INVENTION

The present invention relates to a catalyst carrier, a catalyst, particularly a Fischer-Tropsch catalyst and a method of making the same.

The Fischer-Tropsch process can be used for the conversion of synthesis gas (from hydrocarbonaceous feed stocks) into liquid and/or solid hydrocarbons. Generally, the feed stock (e.g. natural gas, associated gas and/or coal-bed methane, heavy and/or residual oil fractions, coal, biomass) is converted in a first step into a mixture of hydrogen and carbon monoxide (this mixture is often referred to as synthesis gas or syngas). The synthesis gas is then fed into one or more reactors where it is converted in one or more steps over a suitable catalyst at elevated temperature and pressure into paraffinic compounds ranging from methane to high molecular weight modules comprising up to 200 carbon atoms, or, under particular circumstances, even more.

Numerous types of reactor systems have been developed for carrying out the Fischer-Tropsch reaction. For example, Fischer-Tropsch reactor systems include fixed bed reactors, especially multi-tubular fixed bed reactors, fluidised bed reactors, such as entrained fluidised bed reactors and fixed fluidised bed reactors, and slurry bed reactors such as three-phase slurry bubble columns and ebullated bed reactors.

Preferably, a Fischer-Tropsch catalyst is used, which yields substantial quantities of paraffins, more preferably substantially unbranched paraffins. Fischer-Tropsch catalysts are known in the art, and frequently comprise, as the catalytically active component, a metal from Group VIII of the Periodic Table. (References herein to the Periodic Table relate to the previous IUPAC version of the Periodic Table of Elements such as that described in the 68^{th} Edition of the Handbook of Chemistry and Physics (CPC Press)). Particular catalytically active metals include ruthenium, iron, cobalt and nickel. Cobalt and iron are preferred, especially cobalt.

The metal is typically supported on a catalyst carrier that can be a porous refractory oxide, particularly titania. The carrier comprises refractory oxide particles with a size that is chosen or manipulated to the most appropriate size. The particle sizes should be small enough to provide a sufficient surface area for the catalytically active component. If the refractory oxide particles are too big, the catalytically active component particles will be too big producing a smaller surface area for the catalysed reaction. However if the refractory oxide particles are too small, the catalytically active component particles will also be too small and often the porosity of the carrier is restricted thus reducing the amount of catalytically active component which can settle in the pores of the catalyst carrier which limits diffusion and encourages secondary agglomeration, both of which are typically unwanted effects. The particle size also has an influence on the mechanical strength of the catalyst carrier particles and any catalyst prepared therefrom. Additionally, the particles size has an influence on the hydrothermal stability of the catalyst carrier particles and any catalyst prepared therefrom.

Therefore the particle size selected is a compromise between these conflicting requirements. It would be advantageous to mitigate or eliminate one or more of the problems set out above.

### SUMMARY OF THE INVENTION

It has now been found that a catalyst carrier having a particle size distribution with a first peak at a first particle size and a second peak at a second particle size is advantageous.

The particle size distribution is the proportion of particles plotted against the size of the particles. A peak is defined herein as having more than 10% of total particle weight at any one limited range of particle size, preferably at least 20%, preferably at least 30%. The peak is defined at the mode of the peak, that is the particle size having top of the peak range. Preferably the range is within 1 standard deviation of the peak mode. For symmetric peaks, the average particle size for a peak is the same as the particle size at the peak mode.

Preferably a first refractory oxide produces the first peak and a second refractory oxide produces the second peak. In an alternative preferable embodiment, a first crystalline phase of titania produces the first peak and a second crystalline phase of titania produces the second peak. Having two such peaks may be referred to as a bi-modal distribution. In a bi-modal or multi-modal distribution, two peaks are defined when there is a low between peaks which is at least 10% less than the smaller of the two peaks.

The particles preferably are crystalline. Preferably the catalyst carrier comprises more than 90 weight percent crystalline material; most preferably more than 90 weight percent crystalline titania. Preferably the crystalline material comprises anatase, rutile and/or brookite crystalline phases of titania.

It has now been found that adjusting the magnitude of the first and/or of the second particle size has an influence on the surface area as well as on the mechanical strength and/or on the hydrothermal stability of the catalyst carrier and of the catalyst or catalyst precursor prepared from the catalyst carrier. In this way the selectivity and/or activity of a catalyst made from said catalyst support may also be improved.

According to a first aspect of the present invention, there is provided a catalyst carrier comprising more than 90 weight percent crystalline titania, calculated on the total weight of the carrier, and having a particle size distribution with a first peak at a first particle size and a second peak at a second particle size, wherein the second particle size is at least 50% larger than the first particle size, and wherein the first particle size is in the range of from 15 to 27 nm, and wherein the second particles size is in the range of from 30 to 42 nm.

The second particle size is preferably more than 60% larger, more preferably more than 70% larger than the first particle size.

Preferably between 40-90 wt % of particles are of the smaller size, more preferably around 50 wt %.

Preferably more than 15% of the crystals in the carrier, calculated on the total number of crystals in the carrier, has a size of less than 10 nm.

### DETAILED DESCRIPTION OF THE INVENTION

An advantage of a titania catalyst carrier according to the first aspect of the present invention, and of a catalyst or catalyst precursor prepared therefrom, is its high mechanical strength. Carrier particles and catalyst (precursor) particles with an unexpectedly high flat plate crushing strength can be obtained. Therefore a reactor tube can be filled up to a high level without the catalyst particles at the bottom collapsing under the load. Also carrier particles and catalyst (precursor) particles with a high abrasion resistance can be obtained.

The size of particles and the particle size distribution can be determined using any suitable technique. Preferably the particle sizes and the particle size distribution are determined using Transmission electron microscopy (TEM), Scanning electron microscopy (SEM) or laser diffraction, more preferably using TEM.

One suitable way to determine the size of crystals in a titania sample, is to disperse the sample in butanol, subject it to ultrasonic vibration, and analyse it using SEM or TEM. A suitable magnification is 500,000.

In a preferred method, a titania sample is dispersed in butanol, subjected to ultrasonic vibration, and then a few droplets are placed onto a copper-grid supported carbon film. When all butanol has been evaporated, the sample is placed in the transmission electron microscope and analysed.

In a preferred method, pictures are taken of TEM images with a magnification of 500,000. Per titania sample preferably 10 to 16 pictures are taken, each at a different location of the sample, which are then analysed using a ruler or image analysis equipment. Preferably the size of at least 100 crystals, more preferably of at least 300 crystals, is determined.

In a highly preferred method, images are taken at a magnification of 500,000 and printed on A4-sized photo quality or other high-resolution paper using a photo quality or other high-resolution printer and then analysed.

In an alternative highly preferred method, images are taken at a magnification of 500,000 and analysed using a computer and software developed for particle size analysis from images.

The particle size distribution may be determined from the size measured for at least 100 crystals, preferably at least 300 crystals.

According to a second aspect of the present invention, there is provided a catalyst carrier comprising more than 90 weight percent crystalline titania, and having a particle size distribution with a first peak at a first particle size and a second peak at a second particle size, wherein the second particle size is at least 50% larger than the first particle size, and wherein the first particle size is in the range of from 35 to 50 nm, preferably 35 to 45 nm, more preferably 35 to 40 nm, and wherein the second particles size is in the range of from 52 to 70 nm, preferably 55 to 60 nm.

The second particle size is preferably more than 60% larger, more preferably more than 70% larger than the first particle size.

Preferably between 40-90 wt % of particles are of the smaller size, more preferably around 50 wt %.

Preferably less than 5% of the crystals in the carrier, calculated on the total number of crystals in the carrier, has a size of less than 10 nm.

An advantage of a titania catalyst carrier according to the second aspect of the present invention, and of a catalyst or catalyst precursor prepared therefrom, is its high hydrothermal stability. Carrier particles and catalyst (precursor) particles with an unexpectedly high hydrothermal stability can be obtained; these are very well resistant against Fischer-Tropsch conditions. Additionally, catalyst particles can be obtained that show a relatively small diffusion limitation; synthesis gas can enter the pores in the catalyst particles relatively easy.

According to a third aspect of the present invention, there is provided a catalyst carrier comprising more than 90 weight percent crystalline titania, and having a particle size distribution with a first peak at a first particle size and a second peak at a second particle size, wherein the second particle size is more than 70% larger than the first particle size, and wherein the first particle size is in the range of from 10 to 50 nm, preferably 20 to 35 nm, and wherein the second particles size is in the range of from 30 to 200 nm, preferably 40 to 150 nm, more preferably 40 to 70 nm.

The second particle size is preferably 75% or more than 75% larger, more preferably 80% or more than 80% larger than the first particle size.

Preferably between 40-90 wt % of particles are of the smaller size, more preferably around 50 wt %.

An advantage of a titania catalyst carrier according to the third aspect of the present invention, and of a catalyst or catalyst precursor prepared therefrom, is its high hydrothermal stability. Carrier particles and catalyst (precursor) particles with an unexpectedly high hydrothermal stability can be obtained; these are very well resistant against Fischer-Tropsch conditions.

The invention also provides a method for preparing a titania catalyst carrier according to the first aspect of the invention, the method comprising:

providing a first catalyst carrier material comprising more than 90 weight percent crystalline titania, and having a particle size distribution with a single peak at a first particle size; wherein the first particle size is in the range of from 15 to 27 nm;

providing a second catalyst carrier material comprising more than 90 weight percent crystalline titania, and having a particle size distribution with a single peak at a second particle size; wherein the second particles size is in the range of from 30 to 42 nm;

wherein the second particle sizes is at least 50% larger, preferably more than 60% larger, more preferably more than 70% larger than the first particle size;

mixing the first and second carrier material resulting in a mixed carrier material having a particle size distribution with a first peak at the first particle size and a second peak at a second particle size.

The invention also provides a method for preparing a titania catalyst carrier according to the second aspect of the invention, the method comprising:

providing a first catalyst carrier material comprising more than 90 weight percent crystalline titania, and having a particle size distribution with a single peak at a first particle size; wherein the first particle size is in the range of from 35 to 50 nm, preferably 35 to 45 nm, more preferably 35 to 40 nm;

providing a second catalyst carrier material comprising more than 90 weight percent crystalline titania, and having a particle size distribution with a single peak at a second particle size; wherein the second particles size is in the range of from 52 to 70 nm, preferably 55 to 60 nm;

wherein the second particle sizes is at least 50% larger, preferably more than 60% larger, more preferably more than 70% larger than the first particle size;

mixing the first and second carrier material resulting in a mixed carrier material having a particle size distribution with a first peak at the first particle size and a second peak at a second particle size.

The invention also provides a method for preparing a titania catalyst carrier according to the third aspect of the invention, the method comprising:

providing a first catalyst carrier material comprising more than 90 weight percent crystalline titania, and having a particle size distribution with a single peak at a first particle size; wherein the first particle size is in the range of from 10 to 50 nm, preferably 20 to 35 nm; wherein the second particles size is in the range of from 30 to 200 nm, preferably 40 to 150 nm, more preferably 40 to 70 nm;

providing a second catalyst carrier material comprising more than 90 weight percent crystalline titania, and having a particle size distribution with a single peak at a second particle size; wherein the second particles size is in the range of from 30 to 200 nm, preferably 40 to 150 nm, more preferably 40 to 70 nm;

wherein the second particle sizes is more than 70% larger, preferably 75% or more than 75% larger, more preferably 80% or more than 80% larger than the first particle size;

mixing the first and second carrier material resulting in a mixed carrier material having a particle size distribution with a first peak at the first particle size and a second peak at a second particle size.

Thus the invention provides a method for using two carrier materials having a certain mono-modal distribution to form a catalyst carrier with a bi-modal distribution.

Alternatively a titania catalyst carrier according to the first, second or third aspect of the invention may be prepared by crystallising amorphous titania in the presence of larger titania crystals, or via a synthesis process in which small and larger crystals are formed.

Thus the invention provides a method of improving the properties of a catalyst carrier comprising preparing a catalyst carrier according to the first, second or third aspect of the invention by crystallising amorphous titania in the presence of larger titania crystals.

In a titania carrier according to the present invention preferably between 40-90 wt % of particles are of the smaller size, more preferably around 50 wt %.

For certain embodiments, an inverse relationship exists between the difference in particle size and the proportion of the particles of the first particle size provided—for an increasing difference in particle size, less particles of the first particle size are required.

Optionally there may be particles with a third particle size. Typically the third particle size is at least 50% larger than the second particle size, more preferably at least 100% larger, even more preferably at least 150% larger.

Thus optionally the catalyst support has a tri-modal distribution.

Further particles having a particle size distribution with a peak at an even larger particle size may be added to the catalyst support. A multi-modal distribution may thus be formed.

The third particle size may be 250-350 nm, preferably around 300 nm.

Preferably the catalyst carrier comprises more than 90 weight percent crystalline titania having a particle size distribution with a first peak at the first particle size and a second peak at a second particle size, and optionally a third peak at a third particle size. Preferably the catalyst carrier comprises anatase, rutile and/or brookite crystalline phases of titania. The titania material with the first, second and optionally third particle size may each independently be one or more of anatase, rutile and brookite crystalline phases of titania.

In certain embodiments the titania causing the first peak is an anatase crystalline phase of titania and the titania causing the second peak is a rutile crystalline phase of titania. The titania causing the third peak, if present, may be the brookite crystalline phase of titania. For certain embodiments the titania causing the first and second, and optionally third, peak are the same type of crystals, for example, they may all be rutile titania.

In especially preferred embodiments the titania causing the first and second peak both comprise rutile and are preferably both essentially rutile.

The density of the carrier may be between 0.5 and 2 gcm⁻³.

The surface area of the carrier is preferably at least 10 m²/g, preferably at least 20 m²/g, optionally up to 100 m²/g.

Catalytically active particles, as the active component are typically added to the catalyst carrier to form a catalyst. The catalytically active material preferably is cobalt. Alternatively the active metal may be iron or another metal.

One preferred catalyst comprises cobalt or iron as catalytically active metal and manganese or zirconium as promoter.

The catalytically active metal is preferably supported on a titania catalyst support as described herein.

The catalytically active metal and the promoter, if present, may be formed with the carrier material by any suitable treatment, such as dispersing or co-milling. Alternatively, impregnation, kneading and extrusion may be used. After deposition of the metal and, if appropriate, the promoter on the support material, the loaded support is typically subjected to drying and/or to calcination at a temperature of generally from 350 to 750° C., preferably a temperature in the range of from 450 to 600° C. The effect of the calcination treatment is to remove chemically or physically bonded water such as crystal water, to decompose volatile decomposition products and to convert organic and inorganic compounds to their respective oxides. After calcination, the resulting catalyst or catalyst precursor is usually activated by contacting it with hydrogen or a hydrogen-containing gas, typically at temperatures of about 200 to 450° C.

The catalyst is preferably used in a Fischer-Tropsch reaction. Thus the present invention provides a method for the production of liquid hydrocarbons from synthesis gas, the process comprising converting synthesis gas into liquid hydrocarbons, and optionally solid hydrocarbons and optionally liquefied petroleum gas, at elevated temperatures and pressures with a catalyst or catalyst support as described herein.

The optimum amount of catalytically active metal present on the support depends inter alia on the specific catalytically active metal. Typically, the amount of cobalt present in the catalyst may range from 1 to 100 parts by weight per 100 parts by weight of support material, preferably from 3 to 50 parts by weight per 100 parts by weight of support material.

The catalytically active metal may be present in the catalyst together with one or more metal promoters or co-catalysts. The promoters may be present as metals or as the metal oxide, depending upon the particular promoter concerned. Suitable promoters include oxides of metals from Groups IIA, IIIB, IVB, VB, VIB and/or VIIB of the Periodic Table, oxides of the lanthanides and/or the actinides. Preferably, the catalyst comprises at least one of an element in Group IVB, VB, VIIB and/or VIII of the Periodic Table, in particular titanium, zirconium, manganese and/or vanadium, especially manganese or vanadium. As an alternative or in addition to the metal oxide promoter, the catalyst may comprise a metal promoter selected from Groups VIIB and/or VIII of the Periodic Table. Preferred metal promoters include rhenium, manganese, iron, platinum and palladium.

The promoter, if present in the catalyst, is typically present in an amount of from 0.001 to 100 parts by weight per 100 parts by weight of support material, preferably 0.05 to 20, more preferably 0.1 to 15. It will however be appreciated that the optimum amount of promoter may vary for the respective elements which act as promoter.

The Fischer-Tropsch process is well known to those skilled in the art and involves synthesis of hydrocarbons from syngas, by contacting the syngas at reaction conditions with a Fischer-Tropsch catalyst.

The synthesis gas can be provided by any suitable means, process or arrangement. This includes partial oxidation and/or reforming of a hydrocarbonaceous feedstock as is known in the art.

Typically the synthesis gas is produced by partial oxidation of a hydrocarbonaceous feed. The hydrocarbonaceous feed suitably is methane, natural gas, associated gas or a mixture of C1-4 hydrocarbons. The feed comprises mainly, i.e. more than 90 v/v %, especially more than 94%, C1-4 hydrocarbons, especially comprises at least 60 v/v percent methane, preferably at least 75 percent, more preferably 90 percent. Very suitably natural gas or associated gas is used. Suitably, any sulphur in the feedstock is removed.

The partial oxidation of gaseous feedstocks, producing mixtures of especially carbon monoxide and hydrogen, can take place according to various established processes. These processes include the Shell Gasification Process. A comprehensive survey of this process can be found in the Oil and Gas Journal, Sep. 6, 1971, pp 86-90.

The oxygen containing gas for the partial oxidation typically contains at least 95 vol. %, usually at least 98 vol. %, oxygen. Oxygen or oxygen enriched air may be produced via cryogenic techniques, but could also be produced by a membrane based process, e.g. the process as described in WO 93/06041. A gas turbine can provide the power for driving at least one air compressor or separator of the air compression/separating unit. If necessary, an additional compressing unit may be used after the separation process, and the gas turbine in that case may also provide at the (re)start power for this compressor. The compressor, however, may also be started at a later point in time, e.g. after a full start, using steam generated by the catalytic conversion of the synthesis gas into hydrocarbons.

To adjust the H₂/CO ratio in the syngas, carbon dioxide and/or steam may be introduced into the partial oxidation process. Preferably up to 15% volume based on the amount of syngas, preferably up to 8% volume, more preferable up to 4% volume, of either carbon dioxide or steam is added to the feed. Water produced in the hydrocarbon synthesis may be used to generate the steam. As a suitable carbon dioxide source, carbon dioxide from the effluent gasses of the expanding/combustion step may be used. The H₂/CO ratio of the syngas is suitably between 1.5 and 2.3, preferably between 1.6 and 2.0. If desired, (small) additional amounts of hydrogen may be made by steam methane reforming, preferably in combination with the water gas shift reaction. Any carbon monoxide and carbon dioxide produced together with the hydrogen may be used in the gasification and/or hydrocarbon synthesis reaction or recycled to increase the carbon efficiency. Hydrogen from other sources, for example hydrogen itself, may be an option.

The syngas comprising predominantly hydrogen, carbon monoxide and optionally nitrogen, carbon dioxide and/or steam is contacted with a suitable catalyst in the catalytic conversion stage, in which the hydrocarbons are formed. Suitably at least 70 v/v% of the syngas is contacted with the catalyst, preferably at least 80%, more preferably at least 90%, still more preferably all the syngas.

The Fischer-Tropsch synthesis is preferably carried out at a temperature in the range from 125 to 350° C., more preferably 175 to 275° C., most preferably 200 to 260° C. The pressure preferably ranges from 5 to 150 bar abs., more preferably from 5 to 80 bar abs.

The Fischer-Tropsch tail gas may be added to the partial oxidation process.

The Fischer-Tropsch process can be carried out in a slurry phase regime or an ebullating bed regime, wherein the catalyst particles are kept in suspension by an upward superficial gas and/or liquid velocity.

Another regime for carrying out the Fischer-Tropsch process is a fixed bed regime, especially a trickle flow regime. A very suitable reactor is a multitubular fixed bed reactor. In addition, the Fischer-Tropsch process may also be carried out in a fluidised bed process.

Products of the Fischer-Tropsch synthesis may range from methane to heavy paraffin waxes. Preferably, the production of methane is minimised and a substantial portion of the hydrocarbons produced have a carbon chain length of a least 5 carbon atoms. Preferably, the amount of C₅₊ hydrocarbons is at least 60% by weight of the total product, more preferably, at least 70% by weight, even more preferably, at least 80% by weight, most preferably at least 85% by weight.

The hydrocarbons produced in the process are suitably C3-200 hydrocarbons, more suitably C4-150 hydrocarbons, especially C5-100 hydrocarbons, or mixtures thereof. These hydrocarbons or mixtures thereof are liquid or solid at temperatures between 5 and 30° C. (1 bar), especially at about 20° C. (1 bar), and usually are paraffinic of nature, while up to 30 wt %, preferably up to 15 wt %, of either olefins or oxygenated compounds may be present.

Depending on the catalyst and the process conditions used in a Fischer-Tropsch reaction, various proportions of normally gaseous hydrocarbons, normally liquid hydrocarbons and optionally normally solid hydrocarbons are obtained. It is often preferred to obtain a large fraction of normally solid hydrocarbons. These solid hydrocarbons may be obtained up to 90 wt % based on total hydrocarbons, usually between 50 and 80 wt %.

A part may boil above the boiling point range of the so-called middle distillates. The term “middle distillates”, as used herein, is a reference to hydrocarbon mixtures of which the boiling point range corresponds substantially to that of kerosene and gasoil fractions obtained in a conventional atmospheric distillation of crude mineral oil. The boiling point range of middle distillates generally lies within the range of about 150 to about 360° C.

The higher boiling range paraffinic hydrocarbons, if present, may be isolated and subjected to a catalytic hydrocracking step, which is known per se in the art, to yield the desired middle distillates. The catalytic hydro-cracking is carried out by contacting the paraffinic hydrocarbons at elevated temperature and pressure and in the presence of hydrogen with a catalyst containing one or more metals having hydrogenation activity, and supported on a support comprising an acidic function. Suitable hydrocracking catalysts include catalysts comprising metals selected from Groups VIB and VIII of the (same) Periodic Table of Elements. Preferably, the hydrocracking catalysts contain one or more noble metals from Group VIII. Preferred noble metals are platinum, palladium, rhodium, ruthenium, iridium and osmium. Most preferred catalysts for use in the hydro-cracking stage are those comprising platinum.

The amount of catalytically active noble metal present in the hydrocracking catalyst may vary within wide limits and is typically in the range of from about 0.05 to about 5 parts by weight per 100 parts by weight of the support material. The amount of non-noble metal present is preferably 5-60%, preferably 10-50%.

Suitable conditions for the catalytic hydrocracking are known in the art. Typically, the hydrocracking is effected at a temperature in the range of from about 175 to 400° C. Typical hydrogen partial pressures applied in the hydrocracking process are in the range of from 10 to 250 bar.

The product of the hydrocarbon synthesis and consequent hydrocracking suitably comprises mainly normally liquid hydrocarbons, beside water and normally gaseous hydrocarbons. By selecting the catalyst and the process conditions in such a way that especially normally liquid hydrocarbons are obtained, the product obtained (“syncrude”) may be transported in the liquid form or be mixed with any stream of crude oil without creating any problems as to solidification and or crystallization of the mixture. It is observed in this respect that the production of heavy hydrocarbons, comprising large amounts of solid wax, are less suitable for mixing with crude oil while transport in the liquid form has to be done at elevated temperatures, which is less desired.

Thus the invention also provides hydrocarbon products synthesised by a Fischer-Tropsch reaction and catalysed by a catalyst on a support as described herein.

The hydrocarbon may have undergone the steps of hydroprocessing, preferably hydrogenation, hydroisomerisation and/or hydrocracking.

The hydrocarbon may be a fuel, preferably naphtha, kerosene or gasoil, a waxy raffinate or a base oil.

Any percentage mentioned in this description is calculated on total weight or volume of the composition, unless indicated differently. When not mentioned, percentages are considered to be weight percentages. Pressures are indicated in bar absolute, unless indicated differently.

### EXAMPLES

### Test Methods; Flat Plate Crushing Strength

Flat plate crushing strength is generally regarded as a test method to measure strength at which catalyst particles collapse. A strength of about 70 N/cm is generally regarded as the minimum strength required for a catalyst material to be used in chemical reactions such as hydrocarbon synthesis, preferably at least 74 N/cm, more preferably at least 100 N/cm, most preferably at least 120 N/cm. The strength can be related to the compressive strength of concrete being tested in a similar test method (i.e. 10 cm cubed sample between plates), but then on a larger scale.

Currently, there is no national or international standard test or ASTM for flat plate crushing strength. However, the “compression test” for concrete, used to measure compressive strength, is well known in the art. Furthermore the general shapes of catalysts or catalyst precursors, for example the shape of extrudates such as cylinders or ‘trilobes’, are well known. The flat plate crushing test strength is independent of product quality in terms of performance in a catalytic reaction.

Naturally, any comparison of flat plate crushing strength must be made between equivalently shaped particles. Usually, it is made between the “top” and “bottom” sides of particles. Where the particles are regularly shaped such as squares, it is relatively easy to conduct the strength tests and make direct comparison. It is known in the art how to make comparisons where the shapes are not so regular, e.g. by using flat plate crushing strength tests.

### Test Methods; Hydrothermal Stability

Hydrothermal stability can be tested by subjecting catalysts for a relatively long time to a high humidity and elevated temperature, and then evaluating any change in mechanical properties and/or catalytic activity.

The hydrothermal stability of the samples described below was tested as follows. First the flat plate crushing strength of the samples was determined. Then the samples were put in an autoclave for 1 week at a relative humidity of 100%, a temperature of 250° C., and a pressure of 20 bar. Then the flat plate crushing strength of the samples was again determined and compared with the initial strength.

### Test Methods; Particle Size Distribution

In the examples, the size of the crystals in titania samples was determined using TEM. Each titania sample was dispersed in butanol and subjected to ultrasonic vibration. Then a few droplets were placed onto a copper-grid supported carbon film. After all butanol was evaporated the sample was placed in the TEM and analyzed. The TEM was performed at a magnification of 500,000.

Per titania sample preferably about 15 pictures were taken, each at a different location of the sample. The images were printed on A4-sized photo quality paper using a photo quality printer. The pictures were analysed using a ruler. The size of at least 300 crystals was determined, and from that information the particle size distribution was determined.

### COMPARATIVE EXAMPLE

A batch of titania with a bi-modal particle size distribution with a first peak at around 36 nm and a second peak at around 51 nm was provided. The second particle size was thus 42% larger than the first particle size.

A cobalt and manganese containing compound was added to this batch. The resulting mixture was extruded, and the resulting extrudates were calcined for one hour at 550° C.

The resulting catalyst particles showed a flat plate crushing strength of 135 N/cm. After 1 week at a RH of 100% at 250° C. and a pressure of 20 bar, the flat plate crushing strength was 80 N/cm.

### Example According to the First Aspect of the Invention

A batch of titania with a bi-modal particle size distribution with a first peak at around 25 nm and a second peak at around 38 nm was provided. The second particle size was thus 52% larger than the first particle size.

A cobalt and manganese containing compound was added to this batch. The resulting mixture was extruded, and the resulting extrudates were calcined for one hour at 550° C.

The resulting catalyst particles showed a flat plate crushing strength of 240 N/cm. After 1 week at a RH of 100% at 250° C. and a pressure of 20 bar, the flat plate crushing strength was 150 N/cm.

Hence, the strength of the catalyst particles was extremely high and the hydrothermal stability was good as compared to the comparative example.

### Example According to the Second Aspect of the Invention

A batch of titania with a bi-modal particle size distribution with a first peak at around 38 nm and a second peak at around 57 nm was provided. The second particle size was thus 50% larger than the first particle size.

A cobalt and manganese containing compound was added to this batch. The resulting mixture was extruded, and the resulting extrudates were calcined for one hour at 550° C.

The resulting catalyst particles showed a flat plate crushing strength of 155 N/cm. After 1 week at a RH of 100% at 250° C. and a pressure of 20 bar, the flat plate crushing strength was 100 N/cm.

Hence, the strength of the catalyst particles was high and the hydrothermal stability was very good as compared to the comparative example.

### Example According to the Third Aspect of the Invention

A batch of titania with a bi-modal particle size distribution with a first peak at around 30 nm and a second peak at around 54 nm was provided. The second particle size was thus 80% larger than the first particle size.

A cobalt and manganese containing compound was added to this batch. The resulting mixture was extruded, and the resulting extrudates were calcined for one hour at 550° C.

The resulting catalyst particles showed a flat plate crushing strength of 190 N/cm. After 1 week at a RH of 100% at 250° C. and a pressure of 20 bar, the flat plate crushing strength was 120 N/cm.

Hence, the strength of the catalyst particles was high and the hydrothermal stability was very good as compared to the comparative example.

1. A catalyst carrier comprising more than 90 weight percent crystalline titania, calculated on the total weight of the carrier, and having a particle size distribution with a first peak at a first particle size and a second peak at a second particle size, wherein the second particle size is at least 50% larger than the first particle size, and wherein the first particle size is in the range of from 15 to 27 nm, and wherein the second particles size is in the range of from 30 to 42 nm.

2. A catalyst carrier according to claim 1, wherein the second particle size is more than 60% larger than the first particle size.

3. A catalyst carrier according to claim 1, wherein between 40-90 wt % of the particles are of the smaller size.

4. A catalyst carrier according to claim 1, wherein more than 15% of the crystals in the carrier, calculated on the total number of crystals in the carrier, has a size of less than 10 nm.

5. A catalyst carrier comprising more than 90 weight percent crystalline titania, calculated on the total weight of the carrier, and having a particle size distribution with a first peak at a first particle size and a second peak at a second particle size, wherein the second particle size is at least 50% larger than the first particle size, and wherein the first particle size is in the range of from 35 to 50 nm, and wherein the second particles size is in the range of from 52 to 70 nm.

6. A catalyst carrier according to claim 5, wherein the second particle size is more than 60% larger than the first particle size.

7. A catalyst carrier according to claim 5, wherein between 40-90 wt % of the particles are of the smaller size.

8. A catalyst carrier according to claim 5, wherein less than 5% of the crystals in the carrier, calculated on the total number of crystals in the carrier, has a size of less than 10 nm.

9. A catalyst carrier comprising more than 90 weight percent crystalline titania, calculated on the total weight of the carrier, and having a particle size distribution with a first peak at a first particle size and a second peak at a second particle size, wherein the second particle size is more than 70% larger than the first particle size, and wherein the first particle size is in the range of from 10 to 50 nm, and wherein the second particles size is in the range of from 30 to 200 nm.

10. A catalyst carrier according to claim 9, wherein the second particle size is at least 75% larger than the first particle size.

11. A catalyst carrier according to claim 9, wherein between 40-90 wt % of the particles are of the smaller size.

12. A catalyst carrier as claimed in claim 1, wherein particles causing the first peak comprise an anatase crystalline phase of titania and the particles causing the second peak comprise a rutile crystalline phase of titania.

13. A catalyst carrier as claimed in claim 1, wherein the particles causing the first and second peak comprise rutile.

14. A catalyst carrier as claimed in claim 1, wherein the particles causing the first and second peak comprise anatase.

15. A catalyst carrier as claimed in claim 1, further comprising a third peak at a third particle size wherein the third refractory oxide is the brookite crystalline phase of titania.

16. A catalyst carrier as claimed in claim 1, wherein the support has a surface area of between 10 m²/g and 100 m²/g.

17. A method for the production of liquid hydrocarbons from synthesis gas, the process comprising converting synthesis gas into liquid hydrocarbons, and optionally solid hydrocarbons and optionally liquefied petroleum gas, at elevated temperatures and pressures with a catalyst or catalyst support as claimed in claim 1.
